(19) United States
(12) Patent Application Publication
(10) Pub. No.: US 2018/0214649 A1
(43) Pub. Date: Aug. 2, 2018

(54) RESPIRATORY THERAPY DEVICE

(71) Applicant: R. Cegla GmbH & Co. KG, Montabaur (DE)

(72) Inventor: Daniel Peller, Biebertal (DE)

(21) Appl. No.: 15/747,464

(22) PCT Filed: Jul. 5, 2016
(86) PCT No.: PCT/EP2016/065804

§ 371 Date: Jan. 25, 2018

(30) Foreign Application Priority Data
Jul. 27, 2015 (DE) 10 2015 214 141.1

(51) Int. Cl. A61M 16/00; A61M 16/20; A61M 16/08

(52) U.S. Cl. CPC A61M 16/024; A61M 16/1055; A61M 16/0883; A61M 16/208

(57) ABSTRACT

A respiratory therapy device comprises a respiratory air channel having a narrowing that has a variable passage cross section, a pressure sensor designed and arranged to detect the value of a pressure prevailing within the respiratory air channel, an adjustment apparatus designed and arranged to change the passage cross section of the narrowing, a control apparatus having a signal input for supplying the pressure value detected by the pressure sensor and a signal output for outputting an adjustment signal to the adjustment apparatus, wherein a portion of the wall defining the respiratory air channel is formed by a resilient hose piece, and wherein the adjustment apparatus acts on the resilient hose piece from the outside in order to change the passage cross section thereof.

The invention relates to a respiratory therapy device.

Respiratory therapy devices are used by patients suffering from chronic lung diseases, to at least one of the following ends: to release bronchial secretions from the bronchi, prevent collapse of the bronchi, strengthen airways, reduce dyspnoea and improve aspiration of the airways as far as to the lung periphery. One example of a chronic lung disease is cystic fibrosis, a recessively inherited metabolic disease and in which the body's own secretions (including the mucus lining the lungs) are secreted from the glands producing them in a much more viscous form than in healthy people.

However, the respiratory therapy device according to the invention can also be used for other lung diseases, for example chronic bronchitis, asthma, other obstructive lung diseases (COPD, etc.) or bronchiectasis, or for problems due to narrowing within the lung (e.g. after a lung transplant as a result of polyp formation, growths or narrowed anastomoses or even for lung cancer). Using a nose adapter, however, it can also be used for ventilation problems in the paranasal sinuses and middle ear.

In respiratory therapy, a distinction is drawn between two fundamentally different categories of respiratory therapy devices. The first category includes respiratory therapy devices having a pressure generating component. For example, a compressor or other suitable pressure generation equipment supplies the patient with air at a higher pressure, meaning that it is not only their bronchi that are widened, but the lung as a whole too. However, pressure generating components can only be used to provide slight resistance to the patient's breathing and/or to generate pressure fluctuations in the respiratory air. The second category of respiratory therapy devices functions entirely without such pressure generation equipment. In these devices, the therapy is based solely on the pressure generated by the patient's lungs. The present invention relates only to respiratory therapy devices of the second category, i.e. respiratory therapy devices without active pressure generation equipment.

In the large majority of respiratory therapy devices, the patient inhales or exhales against resistance. For example, the respiratory therapy devices may operate according to the PEP principle (positive expiratory pressure), in which an oscillating component can also be superimposed on the pressure, as in the OPEP principle (oscillating PEP). This oscillating component generates intrathoracic percussions, which are used to increase the mobility of the secretions in the lungs. More precisely, the aim is to use the pressure fluctuations and vibrations caused by the percussions to make the secretions more fluid and release them so that they can be more easily transported out of the airways by the air flow or expectorated.

Merely for reasons of clarity, the features of the devices operating according to these principles will be explained below, largely using the example of a respiratory therapy device operating according to the PEP principle:

In the flow channel for the respiratory air, conventional respiratory therapy devices operating according to the PEP principle typically comprise a rigid narrowing or one that can be manually adjusted, optionally in a plurality of steps. This narrowing causes a flow resistance against which the patient exhales, thereby leading to increased pressure in the patient's lungs. This pressure widens and stabilises the patient's bronchial system, such that the mucus can be transported out of the lungs by the respiratory air flow.

In practice, however, the problem often arises that the patient is not able to keep their respiratory flow constant from the beginning to the end of the exhalation. The flow is often significantly greater at the beginning of the exhalation than towards the end. This creates difficulties when selecting the rigid narrowing: if too large an opening is selected, the patient may generate a high respiratory flow at the beginning of the exhalation, but as the respiratory flow weakens towards the end of the exhalation, the pressure in the lungs is no longer sufficient to keep the bronchi open, as required for deep exhalation. By contrast, if too small an opening is selected, the pressure is still high enough towards the end of the exhalation to keep the bronchi open and enable deep exhalation, yet it is then impossible at the beginning of the exhalation to generate a sufficient respiratory flow to transport the secretions.

Another problem that constantly occurs in practice is that of hygiene. It goes without saying that the respiratory therapy device has to be cleaned after each therapeutic measure in order to prevent any germs that could endanger the patient's health from remaining thereon; in the case of cystic fibrosis, these include Pseudomonas aeruginosa, Burkholderia cepacia and others.

In light of this, the object of the present invention is to provide a respiratory therapy device that both makes it possible to generate a sufficient respiratory flow for secretion transport at the beginning of the exhalation, and ensures that the bronchi are kept open towards the end of the exhalation, while ensuring the device can be cleaned effectively.

This object is achieved according to the invention by a respiratory therapy device comprising a respiratory air channel having a narrowing that has a variable passage cross section, a pressure sensor designed and arranged to detect the value of a pressure prevailing within the respiratory air channel, an adjustment apparatus designed and arranged to change the passage cross section of the narrowing, a control apparatus having a signal input for supplying the pressure value detected by the pressure sensor and a signal output for outputting an adjustment signal to the adjustment apparatus, wherein a portion of the wall defining the respiratory air channel is formed by a resilient hose piece, and wherein the adjustment apparatus acts on the resilient hose piece from the outside in order to change the passage cross section thereof.

At this juncture, it should be noted that the respiratory air channel has an end facing the patient (hereinafter also referred to as the “patient end”) and an end remote from the patient. When exhaling through the respiratory therapy device, the end remote from the patient forms an outlet end, and the exhalation produces a flow of respiratory air from the patient end towards the outlet end. During inhalation, however, the end remote from the patient forms an inlet end, and the inhalation produces a flow of respiratory air from the inlet end towards the patient end.

The respiratory therapy device according to the invention is designed on the basis of the knowledge that pressure prevailing within the respiratory air channel is in a reproducible relationship with the pressure prevailing within the bronchi. Therefore, by means of the pressure sensor, the control apparatus and the adjustment apparatus, a control loop can be formed that enables the respiratory therapy device according to the invention to generate a desired pressure curve in the bronchi during exhalation and inhalation by changing the passage cross section of the narrowing in the respiratory air channel in a targeted manner according to the pressure detected by the pressure sensor. The respiratory therapy device according to the invention is also designed on the basis of the knowledge that, in this case, it is not the shape of the passage cross section of the narrowing that is critical, but rather merely the flow resistance with which the narrowing resists the patient's breathing. The respiratory therapy device according to the invention uses this to form the narrowing having a variable passage cross section in that a portion of the wall defining the respiratory air channel is made using a resilient hose piece acted on from the outside by the adjustment apparatus. This means that it is only the easily cleaned inner surface of the resilient hose piece that is ever in contact with the respiratory air, not the adjustment apparatus itself, making cleaning the respiratory therapy device according to the invention simpler overall.

At this juncture, it should be pointed out that, in the context of the present invention, the word “resilient” does not necessarily mean that the hose piece has springy restoring properties that make it automatically return to an initial state, in which the passage cross section has a predetermined shape, when the adjustment device stops applying external force thereto. Instead, it is enough for the hose piece to be sufficiently dimensionally stable that it does not collapse under its own weight alone (i.e. without the external force from the adjustment apparatus) and narrow the passage cross section. If the hose piece has springy restoring properties, however, it may be expedient to also provide the adjustment apparatus with spring elements that compensate for at least some of the forces resulting from the springy hose being pressed and the (dynamic) pressure in the respiratory air channel, and which thus reduce the work for the adjustment mechanism.

To aid cleaning of the respiratory therapy device according to the invention, it is proposed that the respiratory air channel is formed in a respiratory air channel assembly that is separate from the rest of the respiratory therapy device but can be connected thereto in an operationally reliable manner.

By way of example, this can be done by the respiratory air channel assembly comprising an attachment in which a portion of the respiratory air channel is formed and which can be connected to a basic unit of the respiratory therapy device in an operationally reliable manner. The resilient hose piece can be attached to the attachment. If desired, the respiratory air channel assembly can further comprise a mouthpiece and/or a nose adapter that can be attached to said attachment. To clean the respiratory therapy device, the respiratory air channel assembly can be separated from the rest of the respiratory therapy device and preferably also broken down into its component parts. The attachment can, for example, be an attachment that can be inserted into an allocated receiving recess in the basic unit. The respiratory therapy device thus comprises just a few components that come into direct contact with the respiratory air. Additionally, these are cost-effective to produce and simple to clean.

In a development of the invention, a branch line in which the pressure sensor is arranged may originate from the respiratory air channel. Advantageously, a first portion of the branch line can be formed by an aperture, starting from the respiratory air channel, in an attachment wall that encloses the respiratory air channel. Additionally or alternatively, a second portion of the branch line can be formed by a circumferential groove being formed in an outer surface of the circumferential wall of a portion of the respiratory air channel, which groove is connected to the respiratory air channel by means of a radial passage and forms a portion of the branch line. This second portion is advantageous in that it increases the length of the branch line and thus reduces the risk of the pressure sensor becoming contaminated with germs. Advantageously, the second portion can be formed in the outer circumferential wall of the attachment. For example, the circumferential groove can be formed by an annular groove, such that, downstream of the first portion formed by the radial passage, the branch line divides into two limbs which preferably each extend over 180° and join back up again later. Alternatively, however, it is also conceivable for the circumferential groove to be a groove that extends over less than 360°, such that its ends are separated by a barrier. This can further extend the length of the branch line. Lastly, according to another alternative, it is even conceivable to provide the outer circumferential wall of the attachment with a groove of a greater length than the circumference of the attachment. This groove can, for example, extend in a helical and/or meandering and/or other suitable manner.

Since the second portion of the branch line is formed by the circumferential groove in cooperation with an opposite wall of the respiratory therapy device basic unit, it is also advantageous for a seal, for example an O-ring, to be provided between the respiratory air assembly and the basic unit both upstream and downstream of the circumferential groove in the longitudinal direction of the respiratory air channel. Additionally or alternatively, in the case of a groove extending in a helical and/or meandering and/or other suitable manner, a sealing cord and/or a specifically shaped sealing element can also be provided between the respiratory air assembly and the basic unit.

Alternatively, the outer surface of the attachment can also be enclosed by a sleeve element that extends at least over the length portion of the attachment in which the groove in the attachment outer surface forming the second portion of the branch line is provided. Preferably, the sleeve element is made of a rubber-elastic material, for example silicone, such that it can seal the second portion of the branch line from the surroundings. Using a sleeve element of this kind brings the advantage of improved hygiene, since all the additional wall portions defining the branch line, i.e. both the attachment and the sleeve element, can be removed from the basic unit and cleaned.

In a development of the invention, the sleeve element can be integral with the resilient hose piece.

A third portion of the branch line, formed in the basic unit of the respiratory therapy device, can be connected to either the first portion of the branch line or, where present, the second portion of the branch line. Advantageously, the pressure sensor can be assigned to this third portion.

If the pressure sensor is arranged on the main circuit board on which the control apparatus is also arranged, it is advantageous for at least part of the third portion of the branch line to be formed by a hose piece that is inserted into a recess in the basic unit, from where it is guided to the pressure sensor. In this way, the hose piece is arranged in the basic unit in a compact manner, while also being able simple to replace, in particular for cleaning purposes. The hose piece can be replaced in a particularly simple manner when it can be accessed from the outside, for example through a flap in the housing of the respiratory therapy device.

However, if the pressure sensor is connected to the control apparatus by means of a cable, the third portion only needs to extend into the basic unit by a small amount. It is even conceivable to arrange the pressure sensor in the basic unit in such a way that it is directly opposite the end of either the first portion or the second portion, removing the need for the third portion. To improve hygiene, the fully wired pressure sensor can also be accessible from the outside through an opening or a flap in the housing. In this way, the sensor could be replaced in a simple manner, for example by the patient themselves. This replacement could, for example, be carried out as part of the annual service of all the respiratory therapy device components important for hygiene purposes, or at shorter intervals as required.

In order to be able to reliably connect the first portion of the branch line to the third portion, it is advantageous—regardless of whether the second portion is present—for both the attachment and the basic unit of the respiratory therapy device to be assigned interacting orientation aids. These orientation aids can be formed, for example, by a protrusion arranged on the attachment or basic unit and a recess arranged in the other part (i.e. the basic unit or attachment). If the additional sleeve element is provided, it is advantageous, in addition or as an alternative, for one of the interacting orientation aids, for example the protrusion or recess, to be arranged on/in the exterior of the sleeve element and for the other orientation aid, for example the recess or protrusion, to be arranged in/on the basic unit.

To increase hygiene, it is proposed in a development of the invention for a filter to be provided in the respiratory air channel and/or in the branch line. The job of this kind of filter is to retain any germs (bacteria, viruses, moulds and similar pathogens) present in the patient's respiratory air and optionally render them harmless, as well as to retain moisture that could condense in the system and thus create the basis for contamination or mould formation.

If the filter is arranged in the respiratory air channel, it is advantageous for the resistance it provides to the breathing to be smaller than the resistance caused by the narrowing having a variable passage cross section. This can ensure that the filter does not prevent the functioning of the respiratory therapy device according to the invention. From a hygiene perspective, it is advantageous if the filter is arranged in a portion of the respiratory air channel facing the patient in relation to the position at which the branch line branches from the respiratory air channel. In this way, the risk of unwanted germs reaching the branch line and possibly even the pressure sensor can be reduced or even completely eliminated.

If the filter is arranged in the branch line, it is advantageous for hygiene reasons for it to be as close as possible to the position at which the branch line branches from the respiratory air channel. This can also reduce or even completely eliminate the risk of unwanted germs reaching the branch line. If the additional sleeve element is provided, the filter can, for example, be held on the attachment by means of the sleeve element.

Regardless of where exactly the filter is arranged, any effects on the detection of the pressure by means of the pressure sensor can be determined by simple calibration measurements and stored as a characteristic diagram in a memory of the control apparatus.

To aid proper breathing, in a development of the invention a non-return valve can be provided in the respiratory air channel. By means of this non-return valve, the patient can further be prevented from inhaling through the respiratory therapy device and thus being infected with germs that may still be present in the respiratory therapy device. Advantageously, the non-return valve can be arranged between the patient end and the branch line, preferably in a portion of the respiratory air channel located between the patient end and the attachment.

Furthermore, the adjustment apparatus can comprise a non-periodically operating adjustment assembly and also, if desired, a periodically operating adjustment assembly.

A non-periodically operating adjustment assembly is sufficient, for example, if no oscillating pressure component needs to be generated by the respiratory therapy device according to the invention, for example when only the PEP principle will be implemented. In this case, a basic embodiment of the respiratory therapy device merely requires the changes in the respiratory air flow to be compensated for by a change in the passage cross section of the narrowing in such a way as to produce a substantially constant pressure curve. For this, a relatively slow response to changes in the pressure detected by the pressure sensor is sufficient.

In an enhanced embodiment, the respiratory therapy device according to the invention can also be designed to react to coughs and other patient reactions to the respiratory therapy and to thus compensate for resultant pressure changes. For this purpose, it is necessary to have a fast response to changes in the pressure detected by the pressure sensor. Thus, potentially damaging pressure peaks that can occur during the therapy due to incorrect usage can also be prevented and the patient can consequently be protected against injury.

Lastly, it is also conceivable for the non-periodically operating adjustment assembly to also be responsible for the changes in the passage cross section of the narrowing that are necessary for forming the oscillating pressure component and, for example, for implementing the OPEP principle. Since the frequency of this oscillating pressure component should be approximately the same level as the resonance frequency of the ribcage, which is between around 12 Hz and 30 Hz, this requires a non-periodically operating adjustment assembly that can represent changes in the passage cross section of the narrowing at a frequency of around 1 kHz. For this purpose, linear actuators, in particular linear motors, can be used.

By means of a fast-reaction adjustment apparatus of this kind, the pressure level, i.e. the pressure value averaged over an oscillation period, and/or the frequency of the oscillation and/or the amplitude of the oscillation and/or a maximum permissible pressure value and/or a minimum permissible pressure value can be selected freely, in particular independently of the flow of respiratory air.

By means of a fast-reaction adjustment apparatus of this kind, it is also possible to reproduce predetermined pressure curves. This is particularly advantageous for the following reason:

One problem that occurs again and again in practice is that, depending on the patient's state on the day and on how and where the secretions are stuck within the airways, no single conventional respiratory therapy device can bring about the desired therapy effect alone. Therefore, during a therapeutic measure, a plurality of conventional respiratory therapy devices—each having different features, in particular each having a different effect on the pressure curve—may have to be used alternately in order to achieve as good a therapy result as possible or in order to move the secretions as effectively as possible. For this to be possible, patients must of course keep at hand an accordingly large number of respiratory therapy devices, entailing associated costs. Examples of such respiratory therapy devices are disclosed in EP 0 337 990 B1 and DE 44 16 575 A1. In addition, patients must learn how to handle and set up each device in order to ensure the desired therapy result. Lastly, for hygiene reasons, each respiratory therapy device used once has to be cleaned following the therapeutic measure. This entails large amounts of work for patients, especially in respect of those respiratory therapy devices having an adjustable narrowing, in which it is necessary to clean the parts of the adjustment mechanism that come into contact with the respiratory air, and a huge daily burden, in particular for severely ill and weak patients. By means of the respiratory therapy device according to the invention, the characteristic pressure curves of various conventional respiratory therapy devices can now be simulated, stored as control programmes in a memory of the control apparatus, and retrieved as required by using a display apparatus and an input apparatus of the control apparatus. Patients thus require only one single respiratory therapy device during a therapeutic measure.

In this context, it should also be pointed out that “blowing games”, in which the patient controls the game by their breathing, can also be stored in the memory of the control apparatus. This is particularly advantageous for children and teenagers in order to motivate them to regularly carry out their respiratory therapy.

In addition to the non-periodically operating adjustment assembly, a periodically operating adjustment assembly can also be provided. This periodically operating adjustment assembly can provide the oscillating pressure component, meaning that the non-periodically operating adjustment assembly has to meet fewer requirements in terms of response speed. Advantageously, it is possible to adjust the frequency and/or amplitude of the change in the passage cross section of the narrowing in the respiratory air channel caused by the periodically operating adjustment assembly.

In a structurally simple manner, the non-periodically operating adjustment assembly and/or the periodically operating adjustment assembly can comprise an adjustment lever that can be pivoted by means of a servomotor and comprises an engagement element engaged with the resilient hose piece.

In this case, the driven shaft of the servomotor and/or the pivot axis of the adjustment lever can preferably extend substantially orthogonally to the longitudinal extension direction of the respiratory air channel. Furthermore, it is advantageous for the adjustment lever to extend adjacently to the respiratory air channel and substantially in parallel with the respiratory air channel, apart from deviations due to the pivot movement. By means of this assembly, the engagement surfaces that clamp the resilient hose piece between them (whether, for example, on the engagement element of the adjustment lever and a wall portion of the basic unit opposite said lever, or on the engagement elements of two opposite adjustment levers) can always extend substantially in parallel with one another. This can ensure that the resilient hose piece can also be completely clamped when required, and the adjustment force required for complete clamping can be reduced.

The longer the adjustment lever, the smaller the change in the angle that the adjustment lever forms together with the longitudinal extension direction of the respiratory air channel, and the smaller the position change that the engagement element travels between a position corresponding to the maximum passage cross section of the respiratory air channel and a position corresponding to the completely clamped respiratory air channel. The effect of this position change can be reduced if the engagement element tapers towards the resilient hose piece. The taper of the engagement element is also advantageous in that less force is needed to press the hose piece down. Furthermore, the course of the engagement element surface in contact with the hose piece can be shaped such that less force is needed to completely close the passage in the hose piece than with a straight contact surface. Lastly, a rounded engagement surface can prevent damage to the resilient hose piece.

The adjustment lever can be pivoted by means of the servomotor by, for example, the servomotor being connected to the adjustment lever by means of a connecting rod, one end of which is connected to the servomotor eccentrically in relation to its driven shaft and the other end of which is hinged to the adjustment lever.

To produce the non-periodically operating adjustment assembly, the servomotor can be fitted to the basic unit in an operationally reliable manner.

To produce the periodically operating adjustment assembly, it is proposed that the point at which the connecting rod is hinged to the adjustment lever is arranged on the adjustment lever so as to be movable in the longitudinal direction of the adjustment lever, for example slidably received in a slot in the adjustment lever. Furthermore, the servomotor can be fitted in an operationally reliable manner to an auxiliary lever, the position of which relative to the basic unit can be adjusted, for example by means of an additional servomotor.

To form the adjustment lever, it should also be noted that it can be formed having a substantially U-shaped or substantially H-shaped cross section. These cross-sectional shapes produce a particularly sturdy adjustment lever design, in particular when the two side legs of the U-shape or H-shape are arranged on either side of the resilient hose piece at least in portions.

Lastly, in a development of the invention, the control apparatus can also comprise a data transmission interface. By means of this data transmission interface, for example, control programmes for characteristic pressure curves of conventional respiratory therapy devices or pressure curves optimised for the specific patient and/or disease can be entered into the memory of the control apparatus. By means of this interface, many patients can also play the aforementioned “blowing games” with or against one another. Finally, it is also conceivable to use the data transmission interface to transmit to a doctor data, recorded in the memory unit, on the progress of the therapeutic measure. The interface can be designed as a wired interface, e.g. as a USB interface, an optical interface, e.g. an infrared interface, or a radio interface, e.g. a Bluetooth interface.

On the basis of the above discussion of the possibilities for using the respiratory therapy device according to the invention, it is clear that said device can also be used for spirometry measurements, in which the respiratory flow rate and, as a time integral thereof, the respiratory volume are measured as a function of time. The respiratory flow rate can be determined from the measurement of the pressure prevailing within the respiratory air channel on the basis of the fact that a particular position of the servomotors always results in a bijective flow resistance.

In expiratory spirometry, after normal resting breathing, the patient exhales (expires) as much as possible and then inhales (inspires) as much as possible; the difference forms the inspiratory vital capacity (VC). Next, the patient exhales as quickly as possible from a state of full inspiration. The volume exhaled in one second forms the absolute forced expiratory volume in the first second (FEV₁=capacity in one second); the maximum expired volume is called the forced vital capacity (FVC). The ratio of FEV₁/FVC is called the relative capacity in one second FEV₁%.

Using the respiratory therapy device according to the invention, inspiratory measurements can also be carried out. For example, inspiratory pressure PI, in general, or, more specifically, closed-mouth pressure PI_{0.1} 100 milliseconds after inspiration begins and the maximum inspiratory pressure PIₘₐₓ can be determined.

When measuring PI_{0.1}, at the beginning of inhalation after previous normal inhalation and exhalation, the respiratory air channel is briefly closed and the inspiratory pressure 100 milliseconds after the beginning of inhalation is determined. The highest inspiratory pressure determined during the closure is the PIₘₐₓ value. Once the respiratory air channel is opened, breathing continues unhindered. The two values PI_{0.1} and PIₘₐₓ give information on the strength of the respiratory muscles.

In particular, the PIₘₐₓ value can be used as a reference for adjusting the passage cross section of the respiratory air channel when the respiratory therapy device according to the invention is to be used as an inhalation trainer for strengthening the respiratory muscles. Preferably, this inhalation training is carried out at a PI value that is 30% of the PIₘₐₓ value.

Similarly, the maximum exhalation pressure PEₘₐₓ can also be determined, which can be used to regulate the optimum PEP setting over the exhalation flow measured in each case.

It goes without saying that the measurement parameters discussed above, as well as others, can also be input into the respiratory therapy device according to the invention by means of the aforementioned data transmission interface, in order to then be able to be used in the respiratory therapy or the training for the muscles responsible for inhalation and/or exhalation.

It should also be added that the respiratory therapy device according to the invention can be used in any body position. This makes it possible to also be used in stretched-out positions or other body positions advantageous for moving secretions.

At this juncture, reference should again be made to the ease of dismantling and cleaning of the respiratory therapy device according to the invention. In addition, the compact construction of the respiratory therapy device according to the invention allows it to be easily taken on trips, for example as a mobile, rechargeable or battery-operated handheld device. It is important in this respect that the attachment is used both for an operationally reliable connection to the basic unit of the device, and as a sealed portion of the branch line leading to the pressure sensor.

The invention will now be described in the following using a number of embodiments and on the basis of the accompanying drawings, in which:

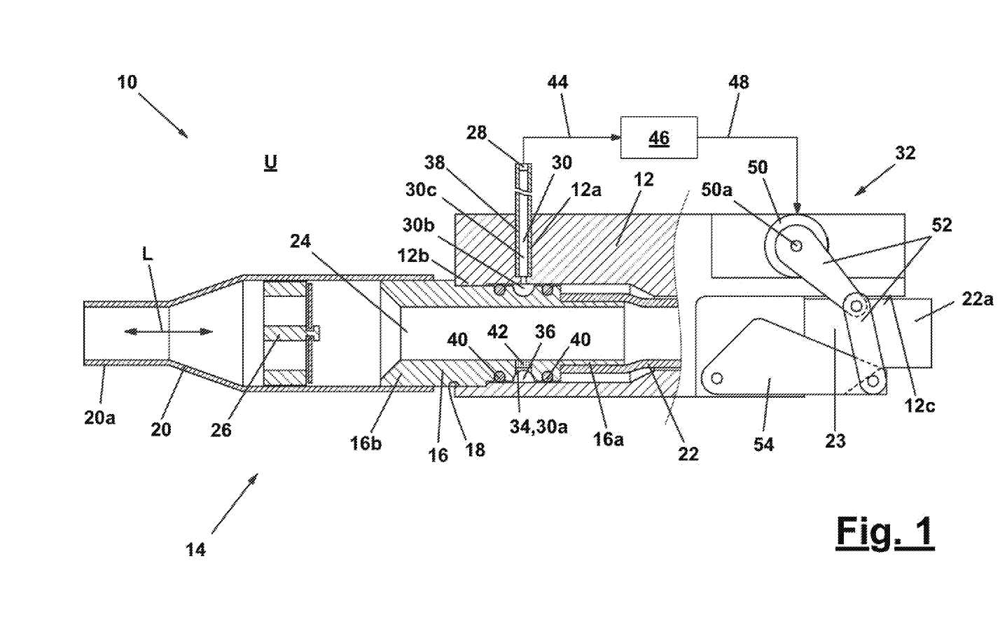

FIG. 1 is a schematic view of a first embodiment of the respiratory therapy device according to the invention;

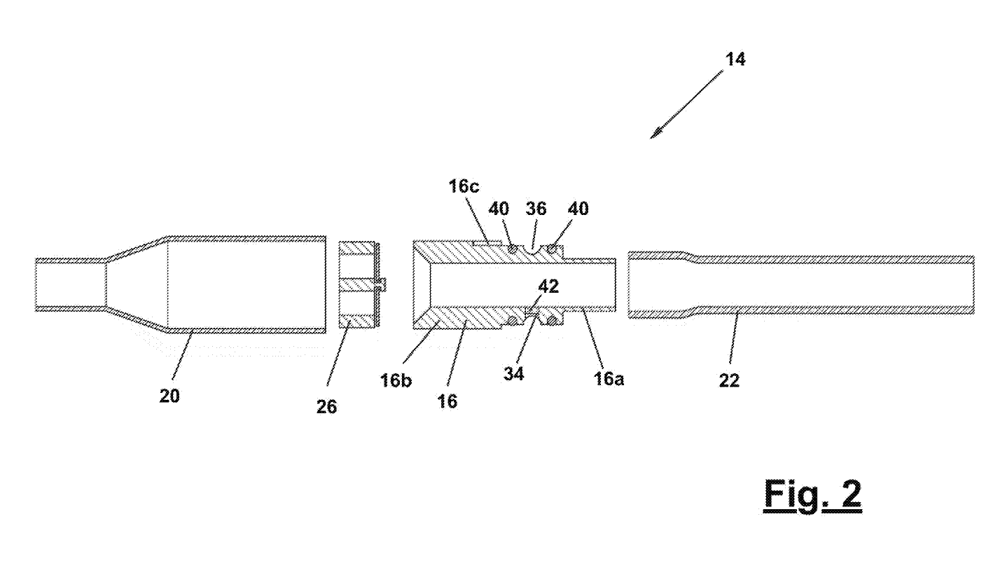

FIG. 2 is an exploded view of the respiratory air channel assembly of the respiratory therapy device in FIG. 1;

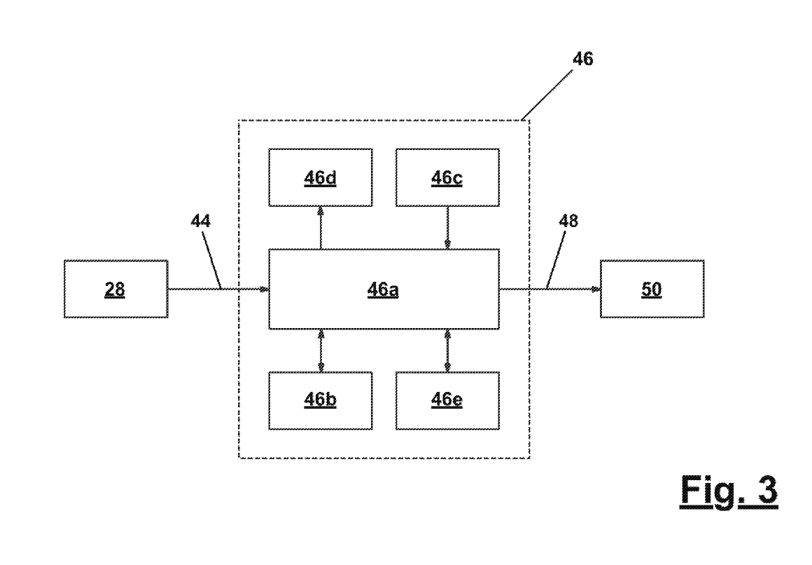

FIG. 3 is a block diagram of the control apparatus of the respiratory therapy device according to the invention;

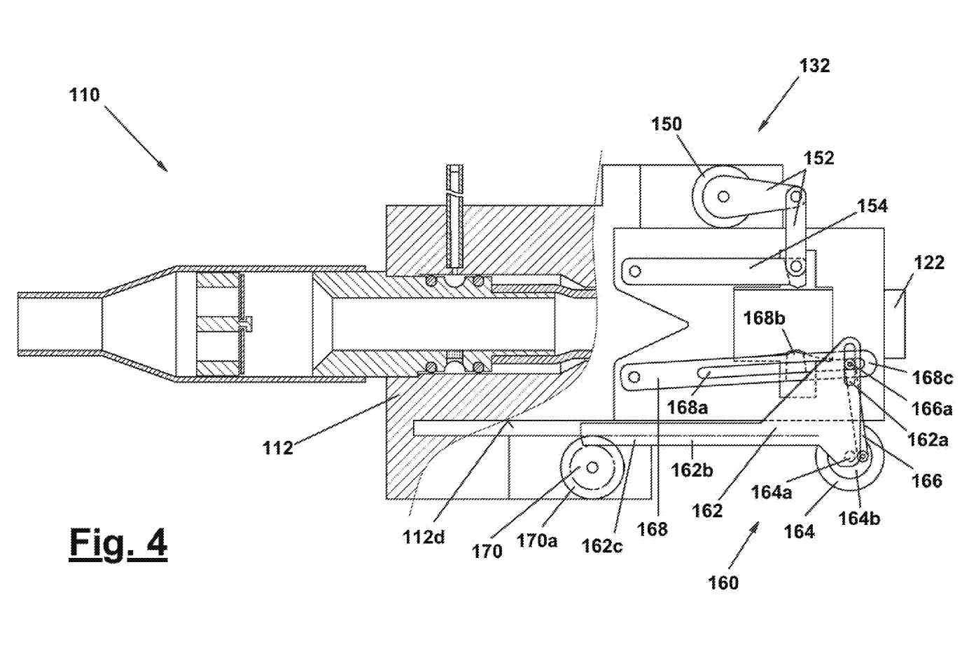

FIG. 4 is a view similar to FIG. 1 of a second embodiment of the respiratory therapy device according to the invention;

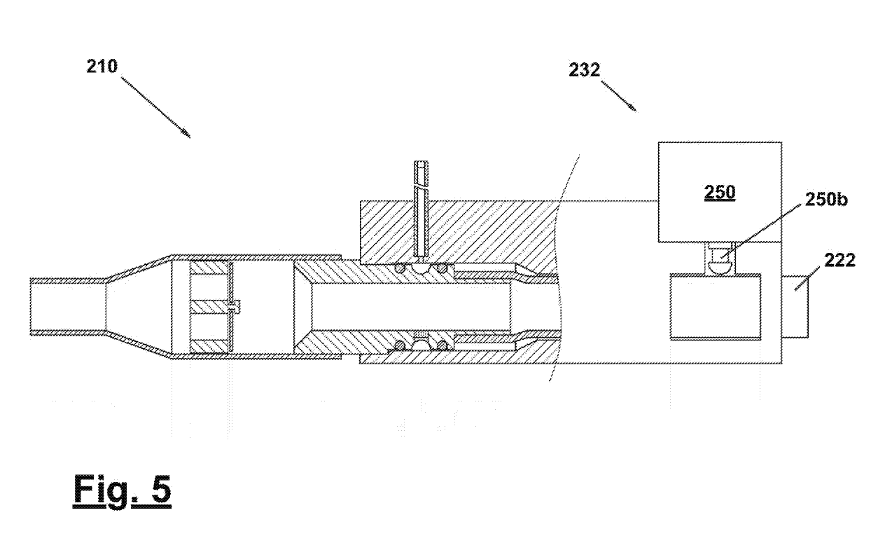

FIG. 5 is a view similar to that in FIGS. 1 and 4 of a third embodiment of the respiratory therapy device according to the invention;

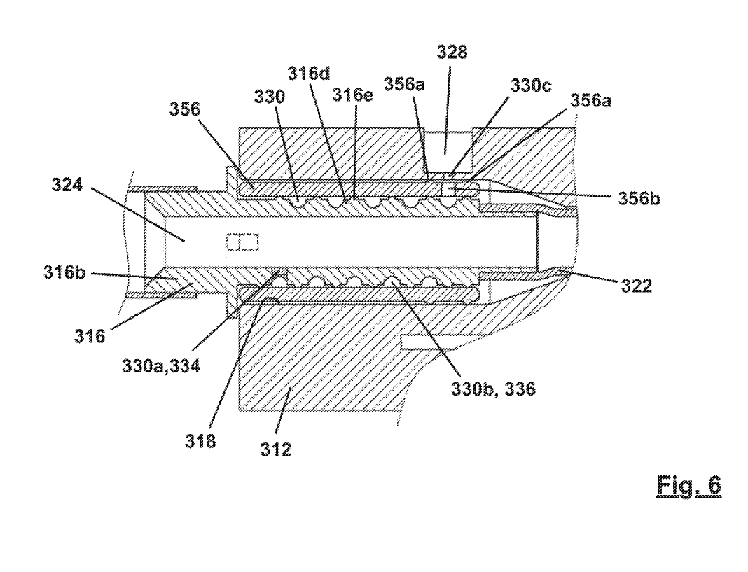

FIG. 6 shows a variant that can be used in the first to third embodiments of the respiratory therapy device according to the invention in accordance with FIGS. 1 to 5; and

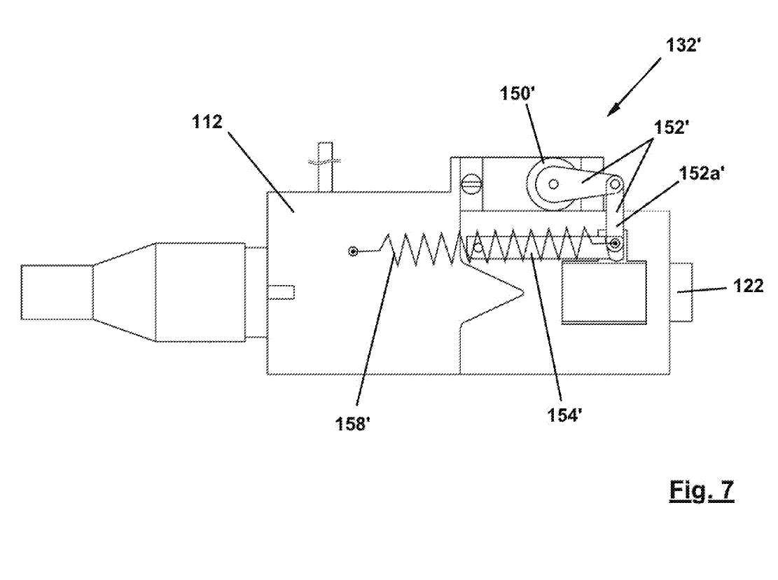

FIG. 7 shows a further variant of the second embodiment of the respiratory therapy device according to the invention as shown in FIG. 4.

FIG. 1 shows a respiratory therapy device according to the invention, generally denoted by 10. The therapy device 10 comprises a basic unit 12 and a respiratory air channel assembly 14, which is connected to the basic unit 12 in an operationally reliable manner.

The respiratory air channel assembly 14 comprises an attachment or insert 16 which, during operation of the respiratory therapy device 10, is inserted into an allocated receiving recess 18 in the basic unit 12 and connected to said unit in an operationally reliable manner. On the side of the attachment 16 facing the patient, a mouth piece 20 is placed over the attachment 16 and connected thereto with a frictional fit and in an air-tight manner. On the side remote from the patient, a resilient hose 22 is placed over a connection piece 16a of the attachment 16 and connected thereto with a frictional fit and in an air-tight manner while widening the hose 22. The respiratory air channel 24 thus extends from a tapered portion 20a of the mouthpiece 20 through the mouthpiece 20, attachment 16 and hose 22 as far as to the end 22a of the hose 22 that is free in FIG. 1.

It should also be pointed out that there is a non-return valve 26, which, if desired, can be arranged in the respiratory air channel 24 in order to set the breathing direction. In the embodiment shown in FIG. 1, the respiratory therapy device 10 operates according to the PEP principle. The non-return valve 26 is therefore designed and arranged such that it opens upon exhalation yet closes during inhalation.

By means of a pressure sensor 28 arranged in a branch line 30 that branches from the respiratory air channel 24, the pressure prevailing in the respiratory air channel 24 can be detected in order to influence the passage cross section of the hose 22 by means of an adjustment apparatus 32 on the basis of the detected pressure in such a way that the pressure within the respiratory air channel 24 and thus also in the patient's lungs is kept substantially constant. The design and functioning of this adjustment apparatus 32 will be discussed further below. In practice, the value of the pressure in the respiratory air channel 24 should be between approximately 2 and 35 hPa, preferably between approximately 4 and 25 hPa, above the air pressure prevailing in the surroundings U.

To prevent the pressure sensor 28 being contaminated with germs possibly contained in the patient's respiratory air, it is advantageous for the branch line 30 to have a predetermined minimum length. In order to still be able to house the branch line within the respiratory therapy device 10, the branch line 30 in the embodiment shown in FIG. 1 comprises a plurality of portions. A first portion 30a is formed by a radially extending aperture 34 in the wall 16b of the attachment 16 that encloses the respiratory air channel 24. A second portion 30b is formed by an annular groove 36 arranged in the outer circumferential surface of the wall 16b. A third portion 30c of the branch line 30 is formed in the basic unit 12 of the respiratory therapy device 10. In the embodiment shown, this third portion 30c is formed by a tube or hose portion 38 inserted into a hole 12a in the basic unit 12. The pressure sensor 28 is arranged at the end of the third portion 30c. Since the tube or hose portion 38 in FIG. 1 protrudes out of the basic unit 12 and is shown in a discontinuous manner, it should be pointed out that the pressure sensor 28 can be arranged in a position remote from the basic unit 12. For example, it can be arranged on the same circuit board on which the control apparatus 46 is also arranged.

As shown in FIG. 1, the aperture 34 opens into the annular groove 36 at a circumferential position that is diametrically opposite the position at which the third portion 30c opens into the annular groove 36. In this way, the second portion 30b of the branch line 30, formed by the annular groove 36, comprises two limbs that separate from one another when the aperture 34 opens into the annular groove 36 and come back together again where the third portion 30c opens. To ensure that the two limbs extend over a circumferential angle of substantially 180°, the attachment 16 and the basic unit 12 are formed having interacting orientation aids. In the embodiment shown, a protrusion 12b on the basic unit 12 engages a recess 16c (see FIG. 2) in the attachment 16.

It should be added that two sealing elements 40 are provided upstream and downstream of the annular groove 36 in the longitudinal direction L of the respiratory air channel 24. These sealing elements 40 are responsible for sealing the branch line 30 from the surroundings U. The sealing elements 40 compressed between the attachment 16 and the basic unit 12 are also used to connect the attachment 16 and basic unit 12 in an operationally reliable manner.

It should also be noted that a filter 42 can be arranged in the aperture 34 and is responsible for keeping any germs possibly contained in the respiratory air of the patient and moisture away from the branch line 30 and thus also away from the pressure sensor 28.

The detection signal of the pressure sensor 28 is supplied via a signal line 44 of a control apparatus 46, in particular the microprocessor 46a thereof (see also FIG. 3). Any pressure drop between the respiratory air channel 24 and the branch line 30 caused by the filter 42 and which could affect the detection result of the pressure sensor 28 can, for example, be compensated for by calibration or comparison measurements with and without the filter 42; the results of these measurements can be stored in a memory 46b of the control apparatus 46 in the form of a characteristic diagram and taken into account by the microprocessor 46a when evaluating the detection signal supplied via the signal line 44.

By taking the detection signal of the pressure sensor 28 into account, the control apparatus 46, or more precisely its microprocessor 46a, determines an adjustment signal for the adjustment apparatus 32 and transmits this to a servomotor 50 of the adjustment apparatus 32 via a signal line 48.

A lever mechanism 52 that acts on an adjustment lever 54 formed in a U-shape in this embodiment is connected to an output shaft 50a of the servomotor 50. The hose 22 is arranged between the side legs of the U-shape of the adjustment lever 54 and is pressed against a bearing surface 12c of the basic unit 12 of the respiratory therapy device 10 by the base leg of the U-shape. If the output shaft 50a of the servomotor 50 in FIG. 1 is turned anti-clockwise, the lever mechanism 52 raises the end of the adjustment lever 54 that is on the right-hand side in FIG. 1, such that the passage cross section of a narrowing 23 in the hose 22 is reduced. The result of this is that the hose 22 resists the patient's breathing to a greater extent, causing the pressure in the respiratory air channel and thus also in the patient's lungs to increase. If, however, the output shaft 50a of the servomotor 50 in FIG. 1 is turned clockwise, the lever mechanism 52 lowers the end of the adjustment lever 54 that is on the right-hand side in FIG. 1, meaning that the passage cross section of the narrowing 23 in the hose 22 increases again due to its own resilience. The result of this is that the hose 22 resists the patient's breathing to a lesser extent, causing the pressure in the respiratory air channel 24 and thus also in the patient's lungs to decrease. In this way, the flow resistance of the respiratory air channel 24 can be influenced in such a way that the pressure in the respiratory air channel 24 and thus also in the patient's lungs can be kept at a substantially constant value.

At the start of a therapeutic measure, the patient activates the respiratory therapy device 10 by means of an on-off button on the input unit 46c of the control apparatus 46, and uses the input unit 46c to select a pressure value at which to set the pressure in the respiratory air channel 24 above the air pressure prevailing in the surroundings U. This pressure value is displayed on a display unit 46d of the control apparatus 46. Next, the patient can begin the therapeutic measure. From the curve of the pressure values from the pressure sensor 28 that the control apparatus 46 continuously monitors, said apparatus can determine when the patient began to exhale through the respiratory air channel 24 and can thus begin to regulate the pressure. In principle, however, it is also conceivable for the pressure regulation to be triggered by pressing a separate button.

During the therapeutic measure, the patient can be given messages by means of the display unit 46d to help them carry out the therapeutic measure. For example, they can be shown information on the pressure value detected at that time and/or on the uniformity of the respiratory air flow, which can be derived from the size and frequency of the adjustment actions by the adjustment apparatus 32.

From this type of display, it is also possible to develop “blowing games” that the patient can play during the therapeutic measure. “Blowing games” of this kind can help motivate children and teenagers to carry out the therapeutic measures that are important for them, but which are often considered bothersome and “uncool”. For example, a “blowing game” of this kind could involve awarding points for the duration and uniformity of the exhalation.

If the control apparatus also comprises a data transmission interface 46e, a plurality of patients can also play the “blowing games” with or against one another.

FIG. 4 shows a second embodiment of a respiratory therapy device according to the invention, which largely corresponds to the embodiment in FIGS. 1 to 3. In FIG. 4, therefore, similar parts are provided with the same reference numerals as in FIGS. 1 to 3, although increased by 100. In addition, the respiratory therapy device 110 in FIG. 4 will be described below only insofar as it differs from the respiratory therapy device 10 in FIGS. 1 to 3, explicit reference otherwise being made to the description thereof.

Unlike the respiratory therapy device 10 in FIGS. 1 to 3, the adjustment apparatus of the respiratory therapy device 110 in FIG. 4 not only comprises a non-periodically operating adjustment assembly 132, but also a periodically operating adjustment assembly 160. In addition, the non-periodically operating adjustment assembly 132 has a different construction from the non-periodically operating adjustment assembly 32 from the embodiment in FIGS. 1 to 3.

Moreover, in the embodiment in FIG. 4, the non-periodically operating adjustment assembly 132 also comprises a servomotor 150 having a lever mechanism 152 that interacts with an additional lever 154, the free end of which presses against the hose 122. However, the additional lever 154 is arranged above the hose 122 in order to create installation space below the hose 122 for the periodically operating adjustment assembly 160.

The periodically operating adjustment assembly 160 comprises a support carriage 162 that carries a servomotor 164. Furthermore, on the driven shaft 164a of the servomotor 164 a disc 164b is in turn arranged, to which a connecting rod lever 166 is hinged eccentrically in relation to the driven shaft 164a. The free end 166a of the connecting rod lever 166 is first guided in a slot 162a in the support carriage 162 and secondly in a slot 168a in another lever 168, which presses against the hose 122 from below using a protrusion 168b.

If the servomotor 164 is rotated, the protrusion 168b presses against the hose 122 periodically. The frequency with which it does this can be varied by means of the speed of the servomotor 164.

To also be able to vary the amplitude of the periodic movement of the protrusion 168b, a ridge 162b on the support carriage 162 has teeth 162c that mesh with teeth 170a of another servomotor 170. The other servomotor 170 is fitted to the basic unit 112 of the respiratory therapy device 110. In addition, the ridge 162b is guided on a guide surface 112d of the basic unit.

If the additional servomotor 170 in FIG. 4 is rotated anticlockwise, the support carriage 162 in FIG. 4 is moved to the left. As a result of the free end 166a of the connecting rod lever 166 being guided in the slot 162a in the support carriage 162, which rises upwards from left to right in FIG. 4, in the process the free end 168c of the additional lever 168 in FIG. 4 is pivoted upwards, such that the protrusion 168b is pressed further against the hose 122. As a result, the amplitude of the periodic movement of the protrusion 168b can be varied.

If, during a rotation of the driven shaft 164a, the speed of the servomotor 164 varies around its nominal speed that determines the frequency of the oscillation, i.e. briefly accelerates and then slows down again, this can not only achieve a sinusoidal oscillation curve, but also any desired oscillation curve. In this way, any pressure profiles can be produced.

FIG. 5 shows a third embodiment of a respiratory therapy device according to the invention, which largely corresponds to the embodiment in FIGS. 1 to 3 and the embodiment in FIG. 4. Therefore, in FIG. 5 similar parts are provided with the same reference numerals as in FIGS. 1 to 3, although increased by 200, or by 100 when compared with FIG. 4. In addition, the respiratory therapy device 210 in FIG. 5 will be described below only insofar as it differs from the respiratory therapy device 10 in FIGS. 1 to 3 and the respiratory therapy device 110 in FIG. 4, explicit reference otherwise being made to the description thereof.

The respiratory therapy device 210 in FIG. 5 differs from the respiratory therapy device 110 in FIG. 4 in that, like the embodiment in FIGS. 1 to 3, it has only one adjustment apparatus, namely the adjustment apparatus 232. Unlike the adjustment apparatus 32 in the embodiment in FIGS. 1 to 3, however, this apparatus does not have a rotating motor, but instead a linear actuator 250, preferably a linear stepper motor, the linearly movable actuator 250b of which presses against the hose 222. Preferably, the response speed of the linear actuator 250 is selected to be so high as to not only depict the non-periodic movements of the embodiments in FIGS. 1 to 4, but also the periodic movements of the embodiment in FIG. 4. In the process, both the frequency and the amplitude of the periodic movement can be varied by actuating the linear actuator 250 accordingly.

FIG. 6 shows a variant that can be used in any of the above-described embodiments of a respiratory therapy device according to the invention. Therefore, in FIG. 6 similar parts are provided with the same reference numerals as in FIGS. 1 to 5, although increased by 300 compared with FIG. 1, by 200 compared with FIG. 4 and by 100 compared with FIG. 5. In addition, the variant in FIG. 6 will be described below only insofar as it differs from the embodiments of FIGS. 1 to 5, explicit reference otherwise being made to the description thereof.

In the variant shown in FIG. 6, the outer surface of the wall 316b of the attachment 316 enclosing the respiratory air channel 324 is enclosed by a sleeve element 356 that extends at least over the length portion of the attachment 316 in which the aperture 334 forming the first portion 330a of the branch line 330 and the groove 336 made in the outer surface of the attachment 316 and forming the second portion 330b of the branch line 330 are located.

While it is in principle conceivable for the sleeve element 356 to be formed of a substantially rigid material, sealing elements corresponding to the sealing elements 40 in the embodiment according to FIG. 1 would then have to be provided in this case, making the overall construction more complex. Preferably, the sleeve element 356 is therefore made of a rubber-elastic material, for example silicone, meaning that it can seal the branch line 330 from the surroundings by being positioned against the outer surface of the attachment 316. For this purpose, ribs 316d which have beads 316e and which separate adjacent portions of the branch line 330 from one another can be formed. When the sleeve element 356 bears against the outer surface of the attachment 316 in a resilient manner, these beads 316e press into the rubber-elastic material of the sleeve element 356 and provide the desired sealing.

In addition, annular ribs 356a can be integrally formed on the outer surface of the sleeve element 356, which ribs enclose an aperture 356b that connects the second portion 330b of the branch line 330 to the third portion 330c of the branch line 330, which leads to the pressure sensor 328, and seal the branch line 330 from the surroundings in cooperation with the inner surface of the receiving recess 318.

In accordance with the above, using the sleeve element 356 made from rubber-elastic material means there is no need for separate sealing elements like the sealing elements 40 in the embodiment of FIG. 1. Using the sleeve element 356 also brings the advantage of improved hygiene, since all the wall portions defining the second portion of the branch line 330, i.e. both the attachment 316 and the sleeve element 356, can be removed from the basic unit 312 and cleaned.

Lastly, it should be noted that it is in principle conceivable for the sleeve element 356 to be integral with the resilient hose piece 322.

FIG. 7 shows a variant of the non-periodically operating adjustment assembly 132 from the embodiment in FIG. 4. Therefore, in FIG. 7 similar parts are provided with the same reference numerals as in FIG. 4, although with an added apostrophe. In addition, the non-periodically operating adjustment assembly 132′ in FIG. 7 will be described below only insofar as it differs from the adjustment assembly 132 in FIG. 4, explicit reference otherwise being made to the description thereof.

According to FIG. 7, the non-periodically operating adjustment assembly 132′ comprises a servomotor 150′ having a lever mechanism 152′ that interacts with an additional lever 154′, the free end of which presses against the hose 122. According to the variant in FIG. 7, a spring element 158′ is also provided (a pretensioned helical tension spring in the embodiment shown), one end of which is connected to the lever 152a′ of the lever mechanism 152′ that is hinged to the additional lever 154′, and the other end of which is hinged to the basic unit 112.

In the position shown in FIG. 7, the spring element 158′ does not exert any force components directed towards the hose 122 on the additional lever 154′. The stability of this position is ensured by the servomotor 150′ or by a gearing connected thereto. The resistance provided thereby is sufficient to hold the additional lever 154′ in the position shown. As soon as the additional lever 154′ is deflected downwards by means of the servomotor 150′ and the lever mechanism 152′ in FIG. 7, the spring element 158′ exerts a force directed towards the hose 122 on the additional lever 154′ and thus assists the servomotor 150′ against the resilient restoring force of the hose 122. Even if the additional lever 154′ moves in a direction that allows the hose 122 to widen, the resilient restoring force of the hose 122 and the force exerted by the spring element 158′ cancel each other out at least partly. The servomotor 150′ can thus be made having lower power overall, meaning more cost-effective servomotors 150′ can be used.

1. Respiratory therapy device comprising:
a respiratory air channel having a narrowing that has a variable passage cross section,
a pressure sensor designed and arranged to detect the value of a pressure prevailing within the respiratory air channel,
an adjustment apparatus designed and arranged to change the passage cross section of the narrowing,
a control apparatus having a signal input for supplying the pressure value detected by the pressure sensor and a signal output for outputting an adjustment signal to the adjustment apparatus,
wherein a portion of the wall defining the respiratory air channel is formed by a resilient hose piece, and
wherein the adjustment apparatus acts on the resilient hose piece from the outside in order to change the passage cross section thereof.

2. Respiratory therapy device according to claim 1, wherein the respiratory air channel is formed in a respiratory air channel assembly that is separate from the rest of the respiratory therapy device but can be connected thereto in an operationally reliable manner.

3. Respiratory therapy device according to claim 2, wherein the respiratory air channel assembly comprises an attachment in which a portion of the respiratory air channel is formed and which can be connected to a basic unit of the respiratory therapy device in an operationally reliable manner.

4. Respiratory therapy device according to claim 1, wherein a branch line in which the pressure sensor is arranged originates from the respiratory air channel.

5. Respiratory therapy device according to claim 4, wherein in an outer surface of the circumferential wall of a portion of the respiratory air channel, there is formed a circumferential groove that is connected to the respiratory air channel by means of a radial passage and forms a portion of the branch line.

6. Respiratory therapy device according to claim 5, wherein a sleeve element is provided and is designed to be positioned against the outer surface of the circumferential wall of the portion of the respiratory air channel and to seal the circumferential groove provided therein from the surroundings.

7. Respiratory therapy device according to claim 1, wherein a filter is provided in the respiratory air channel and/or in the branch line.

8. Respiratory therapy device according to claim 1, wherein a non-return valve is provided in the respiratory air channel.

9. Respiratory therapy device according to claim 1, wherein the adjustment device comprises a non-periodically operating adjustment assembly and also, if desired, a periodically operating adjustment assembly.

10. Respiratory therapy device according to claim 9, wherein the non-periodically operating adjustment assembly and, if desired, the periodically operating adjustment assembly comprise an adjustment lever that acts on the resilient hose piece and can be pivoted by means of a servomotor.

11. Respiratory therapy device according to claim 10, wherein the driven shaft of the servomotor and/or the pivot axis of the adjustment lever extend(s) substantially orthogonally to the longitudinal extension direction of the respiratory air channel.

12. Respiratory therapy device according to claim 9, wherein the adjustment lever is adjacent to the respiratory air channel and extends substantially in parallel with the respiratory air channel.

13. Respiratory therapy device according to claim 10, wherein the non-periodically operating adjustment assembly and, if desired, the periodically operating adjustment assembly comprise a spring element assisting the servomotor.

14. Respiratory therapy device according to claim 1, wherein the control apparatus comprises a data transmission interface.

15. Respiratory therapy device according to claim 1, wherein the control unit is designed to maintain a constant value for the pressure value detected by the pressure sensor and optionally averaged over the duration of an oscillation period, and/or for the amplitude of the oscillation and/or the frequency of the oscillation, or to follow a setpoint.
